(19) United States
(12) Patent Application Publication
(10) Pub. No.: US 2011/0212950 A1
(43) Pub. Date: Sep. 1, 2011

(54) 4,4-DISUBSTITUTED PIPERIDINES

(75) Inventors: Dirk Behnke, Grenzach-Wyhlen (DE); Peter Herold, Munchenstein (CH); Stjepan Jelakovic, Freiburg (DE); Nathalie Jotterand, Basel (CH); Isabelle Lyothier, Huningen (FR); Robert Mah, Muttenz (CH); Stefan Stutz, Basel (CH); Vincenzo Tschinke, Binningen (CH)

(73) Assignee: NOVARTIS AG, Basel (CH)

(21) Appl. No.: 12/734,321

(22) PCT Filed: Oct. 24, 2008
(86) PCT No.: PCT/EP2008/064417

§ 371 Date: May 16, 2011

(30) Foreign Application Priority Data
Oct. 25, 2007 (EP) 07119265.2

(51) Int. Cl. A61K 31/538; C07D 413/12; C07D 413/14; A61P 9/00

(52) U.S. Cl. 514/230.5; 544/105

(57) ABSTRACT

The application relates to 4,4-disubstituted piperidines of the general formula (I) and their salts, preferably their pharmaceutically acceptable salts, in which R², has the meanings explained in the description, a process for their preparation and the use of these compounds as medicines, especially as renin inhibitors.

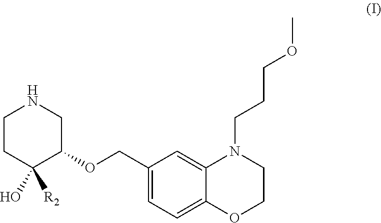

### FIELD OF THE INVENTION

The present invention relates to novel 4,4-disubstituted piperidines, processes for their preparation and the use of the compounds as medicines, in particular as renin inhibitors.

### BACKGROUND OF THE INVENTION

Piperidine derivatives for use as medicines are disclosed for example in WO 97/09311. However, especially with regard to renin inhibition, there continues to be a need for highly potent active ingredients. In this context, the improvement of a compound's pharmacokinetic properties, resulting in better oral bioavailability, and/or it's overall safety profile are at the forefront. Properties directed towards better bioavailability are, for example, increased absorption, metabolic stability or solubility, or optimized lipophilicity. Properties directed towards a better safety profile are, for example, increased selectivity against drug metabolizing enzymes such as the cytochrome P450 enzymes.

### DETAILED DESCRIPTION OF THE INVENTION

The invention therefore relates firstly to trisubstituted piperidines of the general formula

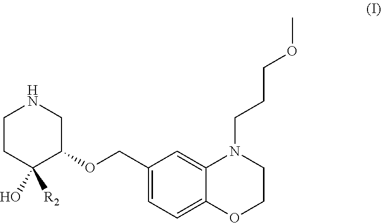

in which

R² is phenyl, which is substituted by 1-3 radicals, one of which is located in the para-position relative to the bond from the phenyl ring to the remainder of the molecule, selected independently from the group consisting of

C₁₋₆-alkanoyloxy-C₁₋₆-alkyl,

C₂₋₆-alkenyl,

C₂₋₆-alkenyloxy,

C₂₋₆-alkenyloxy-C₁₋₆-alkyl,

C₁₋₆-alkoxy,

C₁₋₆-alkoxy-C₁₋₆-alkoxy,

C₁₋₆-alkoxy-C₁₋₆-alkoxy-C₁₋₆-alkoxy,

C₁₋₆-alkoxy-C₁₋₆-alkoxy-C₁₋₆-alkoxy-C₁₋₆-alkyl,

C₁₋₆-alkoxy-C₁₋₆-alkoxy-C₁₋₆-alkyl,

C₁₋₆-alkoxy-C₁₋₆-alkyl,

C₁₋₆-alkoxy-C₁₋₆-alkylamino-C₁₋₆-alkyl,

C₁₋₆-alkoxy-C₁₋₆-alkylsulfanyl,

C₁₋₆-alkoxy-C₁₋₆-alkylsulfanyl-C₁₋₆-alkyl,

C₁₋₆-alkoxycarbonyl,

C₁₋₆-alkoxycarbonyloxy-C₁₋₆-alkyl,

C₁₋₆-alkyl,

C₁₋₆-alkylsulfanyl,

C₁₋₆-alkylsulfanyl-C₁₋₆-alkoxy,

C₁₋₆-alkylsulfanyl-C₁₋₆-alkoxy-C₁₋₆-alkyl,

C₁₋₆-alkylsulfanyl-C₁₋₆-alkyl,

C₁₋₆-alkylsulfonyl-C₁₋₆-alkoxy-C₁₋₆-alkyl,

C₁₋₆-alkylsulfonyl-C₁₋₆-alkyl,

C₂₋₈-alkynyl,

optionally N-mono- or N,N-di-C₁₋₆-alkylated amino-C₁₋₆-alkoxy,

optionally N-mono- or N,N-di-C₁₋₆-alkylated amino-carbonyl-C₁₋₆-alkyl,

aryl-pyrrolidinyl-C₀₋₆-alkoxy,

heterocyclyl-pyrrolidinyl-C₀₋₆-alkoxy,

aryl-C₀₋₆-alkoxy-C₁₋₆-alkoxy,

aryl-C₀₋₆-alkoxy-C₁₋₆-alkoxy-C₁₋₆-alkyl,

carboxy-C₁₋₆-alkyl,

cyano,

cyano-C₁₋₆-alkyl,

C₃₋₈-cycloalkyl-C₀₋₆-alkoxy-C₁₋₆-alkoxy,

C₃₋₈-cycloalkyl-C₀₋₆-alkoxy-C₁₋₆-alkoxy-C₁₋₆-alkyl,

C₃₋₈-cycloalkyl-C₀₋₆-alkoxy-C₁₋₆-alkyl,

C₃₋₈-cycloalkyl-C₀₋₆-alkylamino-C₁₋₆-alkyl,

heterocyclyl-carbonyl-C₁₋₆-alkyl,

heterocyclyl-sulfanyl-C₁₋₆-alkoxy-C₁₋₆-alkyl and

heterocyclyl-C₂₋₆-alkoxy-C₁₋₆-alkyl;

and may, in addition to the aforementioned substituents, also be substituted by a maximum of 2 halogens, the maximum total number of substituents on the phenyl radical of R² being 3;

and the salts thereof, preferably the pharmaceutically acceptable salts thereof.

The meaning of “C₀-alkyl” in the above (and hereinafter) mentioned C₀₋₆-alkyl groups is a bond or, if located at a terminal position, a hydrogen atom.

The meaning of “C₀-alkoxy” in the above (and hereinafter) mentioned C₀₋₆-alkoxy groups is “—O—” or, if located at a terminal position, an —OH group.

C₁₋₆-Alkyl and alkoxy radicals may be linear or branched. Examples of C₁₋₆-alkyl and alkoxy radicals are methyl, ethyl, n-propyl, isopropyl, n-butyl, isobutyl, sec-butyl, tert-butyl, pentyl, hexyl, and methoxy, ethoxy, propoxy, isopropoxy, butoxy, isobutoxy, sec-butoxy and tert-butoxy. C₁₋₆-Alkylenedioxy radicals are preferably methylenedioxy, ethylenedioxy and propylenedioxy. C₁₋₆-alkanoyl refers to C₁₋₆-alkylcarbonyl. Examples of C₁₋₆-alkanoyl radicals are acetyl, propionyl and butyryl.

Cycloalkyl refers to a saturated cyclic hydrocarbon radicals having 3 to 7 carbon atoms, for example cyclopropyl, cyclobutyl or cyclopentyl.

C₁₋₆-Alkylene radicals may be linear or branched and are, for example, methylene, ethylene, propylene, 2-methylpropylene, 2-methylbutylene, 2-methylpropyl-2-ene, butyl-2-ene, butyl-3-ene, propyl-2-ene, tetra-, penta- and hexamethylene; C₂₋₆-alkenylene radicals are, for example, vinylene and propenylene; C₂₋₆-alkynylene radicals are, for example, ethynylene; acyl radicals are alkanoyl radicals, preferably C₁₋₆-alkanoyl radicals, or aroyl radicals such as benzoyl.

Aryl refers to mononuclear aromatic radicals which may be substituted one or more times, such as, for example, phenyl or substituted phenyl, and may be unsubstituted or substituted one or more times, e.g. substituted once or twice by C₁₋₆-alkoxy, C₁₋₆-alkyl, optionally esterified carboxy, cyano, halogen, hydroxy, halogen substituted C₁₋₆-alkoxy, halogen substituted C₁₋₆-alkyl or phenyl.

The term substituted by halogen refers to a substituent such as bromo, chloro, fluoro or iodo.

The term heterocyclyl refers to 3-7 membered monocyclic, saturated, partially unsaturated and maximally unsaturated heterocyclic radicals having 1 to 5 nitrogen and/or 1 or 2 sulfur or oxygen atoms, which may be substituted one or more times, such as, for example, substituted once, twice or three times by C₁₋₆-alkoxy, C₁₋₆-alkoxy-C₁₋₆-alkyl, C₁₋₆-alkyl, aryl, cyano, halogen, heterocyclyl, hydroxy, halogen substituted C₁₋₆-alkoxy or halogen substituted C₁₋₆-alkyl. Heterocyclyl radicals which comprise a nitrogen atom may be linked either via the N atom or via a C atom to the remainder of the molecule.

Examples of such heterocycles are

imidazolyl,

oxetanyl,

pyrazolyl.

pyrrolidinyl,

tetrazolyl,

thiazolyl,

triazolyl.

Heterocyclyl radicals which comprise a nitrogen atom may be linked either via the N atom or via a C atom to the remainder of the molecule.

Hydroxy-substituted C₁₋₆-alkoxy may be for example hydroxy-C₁₋₆-alkoxy or else polyhydroxy-C₁₋₆-alkoxy.

The term halogen-substituted C₁₋₆-alkyl refers to C₁₋₆-alkyl radicals which may be substituted by 1-6 halogen atoms, such as, for example, bromo, chloro, fluoro, iodo. An analogous statement applies to radicals, such as halogen-substituted C₁₋₆-alkoxy.

Salts are primarily the pharmaceutically acceptable or nontoxic salts of compounds of formula (I). The term “pharmaceutically acceptable salts” encompasses salts with inorganic or organic acids, such as hydrochloric acid, hydrobromic acid, nitric acid, sulfuric acid, phosphoric acid, citric acid, formic acid, maleic acid, acetic acid, succinic acid, tartaric acid, methanesulfonic acid, p-toluenesulfonic acid and the like.

Salts of compounds having salt-forming groups are in particular acid addition salts, salts with bases, or, in the presence of a plurality of salt-forming groups, in some cases also mixed salts or internal salts.

Such salts are formed, for example, from compounds of formula (I) with an acidic group, for example a carboxyl or sulfonyl group, and are, for example, the salts thereof with suitable bases such as non-toxic metal salts derived from metals of group Ia, Ib, IIa and IIb of the Periodic Table of the Elements, for example alkali metal, in particular lithium, sodium, or potassium, salts, alkaline earth metal salts, for example magnesium or calcium salts, and also zinc salts and ammonium salts, including those salts which are formed with organic amines, such as optionally hydroxy-substituted mono-, di- or trialkylamines, in particular mono-, di- or tri(lower alkyl)amines, or with quaternary ammonium bases, e.g. methyl-, ethyl-, diethyl- or triethylamine, mono-, bis- or tris(2-hydroxy(lower alkyl))amines, such as ethanol-, diethanol- or triethanolamine, tris(hydroxymethyl)methylamine or 2-hydroxy-tert-butylamine, N,N-di(lower alkyl)-N-(hydroxy(lower alkyl))amine, such as N,N-di-N-dimethyl-N-(2-hydroxyethyl)amine, or N-methyl-D-glucamine, or quaternary ammonium hydroxides such as tetrabutyl ammoniumhydroxide. The compounds of formula (I) having a basic group, for example an amino group, may form acid addition salts, for example with suitable inorganic acids, e.g. hydrohalic acid such as hydrochloric acid, hydrobromic acid, sulfuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulfonic or phosphonic acids or N-substituted sulfamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, and also amino acids, for example the alpha-amino acids mentioned above, and also methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, 2- or 3-phosphoglycerate, glucose 6-phosphate, N-cyclohexylsulfamic acid (with formation of the cyclamates) or with other acidic organic compounds such as ascorbic acid. Compounds of formula (I) having acidic and basic groups may also form internal salts.

Salts obtained may be converted to other salts in a manner known per se, acid addition salts, for example, by treating with a suitable metal salt such as a sodium, barium or silver salt, of another acid in a suitable solvent in which an inorganic salt which forms is insoluble and thus separates out of the reaction equilibrium, and base salts by release of the free acid and salt reformation.

The compounds of formula (I), including their salts, may also be obtained in the form of hydrates or include the solvent used for the crystallization.

For the isolation and purification, pharmaceutically unsuitable salts may also find use.

The compounds of formula (I) also include those compounds in which one or more atoms are replaced by their stable, non-radioactive isotopes; for example a hydrogen atom by deuterium.

The compounds of the formula (I) also include compounds that have been nitrosated through one or more sites such as oxygen (hydroxyl condensation), sulphur (sulphydryl condensation) and/or nitrogen. The nitrosated compounds of the present invention can be prepared using conventional methods known to one skilled in the art. For example, known methods for nitrosating compounds are described in WO 2004/098538 A2.

The compounds of the formula (I) also include compounds that have been converted at one or more sites such that a nitrate-ester-containing linker is attached to an existing oxygen and/or nitrogen. Such “nitroderivatives” of the compounds of the present invention can be prepared using conventional methods known to one skilled in the art. For example, known methods for converting compounds into their nitroderivatives are described in WO 2007/045551 A2.

The compounds of formula (I) have at least two asymmetric carbon atoms and may therefore be in the form of optically pure diastereomers, diastereomeric mixtures, diastereomeric racemates, mixtures of diastereomeric racemates or as meso compounds. The invention encompasses all of these forms. Diastereomeric mixtures, diastereomeric racemates or mixtures of diastereomeric racemates may be separated by customary procedures, for example by column chromatography, thin-layer chromatography, HPLC and the like.

The compounds of formula (I) may also be prepared in optically pure form. The separation into antipodes can be effected by procedures known per se, either preferably at an earlier synthetic stage by salt formation with an optically active acid, for example (+)- or (−)-mandelic acid and separation of the diastereomeric salts by fractional crystallization, or preferably at a relatively late stage by derivatizing with a chiral auxiliary building block, for example (+)- or (−)-camphanoyl chloride, and separation of the diastereomeric products by chromatography and/or crystallization and subsequent cleavage of the bonds to give the chiral auxiliary. The pure diastereomeric salts and derivatives may be analysed to determine the absolute configuration of the piperidine present with common spectroscopic procedures, and X-ray spectroscopy on single crystals constitutes a particularly suitable procedure.

It is possible for the configuration at individual chiral centres in a compound of formula (I) to be inverted selectively. For example, the configuration of asymmetric carbon atoms which bear nucleophilic substituents, such as amino or hydroxyl, may be inverted by second-order nucleophilic substitution, if appropriate after conversion of the bonded nucleophilic substituent to a suitable nucleofugic leaving group and reaction with a reagent which introduces the original substituents, or the configuration at carbon atoms having hydroxyl groups can be inverted by oxidation and reduction, analogously to the process in the European patent application EP-A-0 236 734. Also advantageous is the reactive functional modification of the hydroxyl group and subsequent replacement thereof by hydroxyl with inversion of configuration.

The compound groups mentioned below are not to be regarded as closed, but rather parts of these compound groups may be exchanged with one another or with the definitions given above or omitted in a sensible manner, for example to replace general by more specific definitions. The definitions are valid in accordance with general chemical principles, such as, for example, the common valences for atoms.

The compounds of formula (I) can be prepared in an analogous manner to preparation processes disclosed in the literature. Similar preparation processes are described for example in WO 97/09311 and WO 00/063173. Details of the specific preparation variants can be found in the examples.

Preference is given to compounds of the formula (I) and the salts thereof, preferably the pharmaceutically acceptable salts thereof, in which R² is phenyl, substituted by 1-3 radicals, one of which is located in the para-position relative to the bond from the phenyl ring to the remainder of the molecule, selected independently from the group consisting of

C₁₋₆-alkoxy,

C₁₋₆-alkoxy-C₁₋₆-alkoxy,

C₁₋₆-alkoxy-C₁₋₆-alkoxy-C₁₋₆-alkoxy,

C₁₋₆-alkoxy-C₁₋₆-alkoxy-C₁₋₆-alkoxy-C₁₋₆-alkyl,

C₁₋₆-alkoxy-C₁₋₆-alkoxy-C₁₋₆-alkyl,

C₁₋₆-alkoxy-C₁₋₆-alkyl,

C₁₋₆-alkoxy-C₁₋₆-alkylsulfanyl,

C₁₋₆-alkoxy-C₁₋₆-alkylsulfanyl-C₁₋₆-alkyl,

C₁₋₆-alkyl,

C₁₋₆-alkylsulfanyl-C₁₋₆-alkoxy,

C₁₋₆-alkylsulfanyl-C₁₋₆-alkoxy-C₁₋₆-alkyl,

aryl-pyrrolidinyl-C₀₋₆-alkoxy,

C₃₋₈-cycloalkyl-C₀₋₆-alkoxy-C₁₋₆-alkyl,

heterocyclyl-C₂₋₆-alkoxy-C₁₋₆-alkyl and

heterocyclyl-pyrrolidinyl-C₀₋₆-alkoxy.

R² is particularly preferably

phenyl, substituted by 1-2 radicals, one of which is located in the para-position relative to the bond from the phenyl ring to the remainder of the molecule, selected independently from the group consisting of

C₁₋₆-alkoxy,

C₁₋₆-alkoxy-C₁₋₆-alkoxy,

C₁₋₆-alkoxy-C₁₋₆-alkoxy-C₁₋₆-alkoxy,

C₁₋₆-alkoxy-C₁₋₆-alkoxy-C₁₋₆-alkoxy-C₁₋₆-alkyl,

C₁₋₆-alkoxy-C₁₋₆-alkoxy-C₁₋₆-alkyl,

C₁₋₆-alkoxy-C₁₋₆-alkyl,

C₁₋₆-alkyl,

C₃₋₈-cycloalkyl-C₀₋₆-alkoxy-C₁₋₆-alkyl,

heterocyclyl-C₂₋₆-alkoxy-C₁₋₆-alkyl and

heterocyclyl-pyrrolidinyl-C₀₋₆-alkoxy.

among these, those compounds where a second substituent is present at the phenyl ring, the ones where the second substituent is in the ortho-position relative to the bond from the phenyl ring to the remainder of the molecule are especially preferred

R² is very particularly preferably

phenyl, para-substituted relative to the bond from the phenyl ring to the remainder of the molecule by 1 radical selected from the group consisting of

C₁₋₆-alkoxy,

C₁₋₆-alkoxy-C₁₋₆-alkoxy,

C₁₋₆-alkoxy-C₁₋₆-alkoxy-C₁₋₆-alkoxy,

C₁₋₆-alkoxy-C₁₋₆-alkoxy-C₁₋₆-alkoxy-C₁₋₆-alkyl,

C₁₋₆-alkoxy-C₁₋₆-alkoxy-C₁₋₆-alkyl,

C₁₋₆-alkoxy-C₁₋₆-alkyl,

C₁₋₆-alkyl,

C₃₋₈-cycloalkyl-C₀₋₆-alkoxy-C₁₋₆-alkyl,

heterocyclyl-C₂₋₆-alkoxy-C₁₋₆-alkyl and

heterocyclyl-pyrrolidinyl-C₀₋₆-alkoxy.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S- and O-acyl derivatives of thiols, alcohols or phenols, the acyl group being defined as herein. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower omega-(amino, mono- or dialkylamino, carboxy, lower alkoxycarbonyl)-alkyl esters or such as lower alpha-(alkanoyloxy, alkoxycarbonyl or dialkylaminocarbonyl)-alkyl esters; conventionally, pivaloyloxymethyl esters and similar esters are used as such.

Because of the close relationship between a free compound, a prodrug derivative and a salt compound, a particular compound in this invention also includes its prodrug derivative and salt form, where this is possible and appropriate

The compounds of formula (I) and their pharmaceutically acceptable salts have an inhibitory effect on the natural enzyme renin. The latter passes from the kidneys into the blood and there brings about the cleavage of angiotensinogen to form the decapeptide angiotensin I which is then cleaved in the lung, the kidneys and other organs to the octapeptide angiotensin II. Angiotensin II raises the blood pressure both directly by arterial constriction, and indirectly by releasing the hormone aldosterone, which retains sodium ions, from the adrenals, which is associated with an increase in the extracellular fluid volume. This increase is attributable to the effect of angiotensin II itself or of the heptapeptide angiotensin III formed therefrom as cleavage product. Inhibitors of the enzymatic activity of renin bring about a reduction in the formation of angiotensin I and, as a consequence thereof, the formation of a smaller amount of angiotensin II. The reduced concentration of this active peptide hormone is the direct cause of the blood pressure-lowering effect of renin inhibitors.

The effect of renin inhibitors is detected inter alia experimentally by means of in vitro tests where the reduction in the formation of angiotensin I is measured in various systems (human plasma, purified human renin together with synthetic or natural renin substrate). The following in vitro test of Nussberger et al. (1987) J. Cardiovascular Pharmacol., Vol. 9, pp. 39-44, is used inter alia. This test measures the formation of angiotensin I in human plasma. The amount of angiotensin I formed is determined in a subsequent radioimmunoassay. The effect of inhibitors on the formation of angiotensin I is tested in this system by adding various concentrations of these substances. The IC₅₀ is defined as the concentration of the particular inhibitor which reduces the formation of angiotensin I by 50%. The compounds of the present invention show inhibitory effects in the in vitro systems at minimal concentrations of about 10⁻⁶ to about 10⁻¹⁰ mol/I.

Illustrative of the invention, the compounds of examples 361, 367 and 369-378 inhibit the formation of angiotensin I with IC₅₀ values in the range of about 0.5-800.10·9 mol/I.

| Example No. | IC₅₀ (nM)* |
|---|---|
| 361 | 40.4 |
| 371 | 123.0 |
| 375 | 84.3 |
| 378 | 9.6 |

*A lower inhibiting activity corresponds to a higher IC₅₀ value

Renin inhibitors bring about a fall in blood pressure in salt-depleted animals. Human renin differs from renin of other species. Inhibitors of human renin are tested using primates (marmosets, Callithrix jacchus) because human renin and primate renin are substantially homologous in the enzymatically active region. The following in vivo test is employed inter alia: the test compounds are tested on normotensive marmosets of both sexes with a body weight of about 350 g, which are conscious, unrestrained and in their normal cages. Blood pressure and heart rate are measured with a catheter in the descending aorta and are recorded radiometrically. Endogenous release of renin is stimulated by combining a low-salt diet for 1 week with a single intramuscular injection of furosemide (5-(aminosulfonyl)-4-chloro-2-[(2-furanylmethyl)amino]benzoic acid) (5 mg/kg). 16 hours after the furosemide injection, the test substances are administered either directly into the femoral artery by means of a hypodermic needle or as suspension or solution by gavage into the stomach, and their effect on blood pressure and heart rate is evaluated. The compounds of the present invention have a blood pressure-lowering effect in the described in vivo test with i.v. doses of about 0.003 to about 0.3 mg/kg and with oral doses of about 0.3 to about 30 mg/kg.

The blood pressure-reducing effect of the compounds described herein can be tested in vivo using the following protocol:

The investigations take place in 5 to 6-week old, male double transgenic rats (dTGR), which overexpress both human angiotensinogen and human renin and consequently develop hypertension (Bohlender J. et al., J. Am. Soc. Nephrol. 2000; 11: 2056-2061).

This double transgenic rat strain was produced by crossbreeding two transgenic strains, one for human angiotensinogen with the endogenous promoter and one for human renin with the endogenous promoter. Neither single transgenic strain was hypertensive. The double transgenic rats, both males and females, develop severe hypertension (mean systolic pressure, approximately 200 mm Hg) and die after a median of 55 days if untreated. The fact that human renin can be studied in the rat is a unique feature of this model. Age-matched Sprague-Dawley rats serve as non-hypertensive control animals. The animals are divided into treatment groups and receive test substance or vehicle (control) for various treatment durations. The applied doses for oral administration may range from 0.5 to 100 mg/kg body weight. Throughout the study, the animals receive standard feed and tap water ad libitum. The systolic and diastolic blood pressure, and the heart rate are measured telemetrically by means of transducers implanted in the abdominal aorta, allowing the animals free and unrestricted movement.

The effect of the compounds described herein on kidney damage (proteinuria) can be tested in vivo using the following protocol:

The investigations take place in 4-week old, male double transgenic rats (dTGR), as described above. The animals are divided into treatment groups and receive test substance or vehicle (control) each day for 7 weeks. The applied doses for oral administration may range from 0.5 to 100 mg/kg body weight. Throughout the study, the animals receive standard feed and tap water ad libitum. The animals are placed periodically in metabolism cages in order to determine the 24-hour urinary excretion of albumin, diuresis, natriuresis, and urine osmolality. At the end of the study, the animals are sacrificed and the kidneys and hearts may also be removed for determining the weight and for immunohistological investigations (fibrosis, macrophage/T cell infiltration, etc.).

The pharmacokinetic properties of the compounds described herein can be tested in vivo using the following protocol:

The investigations take place in pre-catheterized (carotid artery) male rats (300 g±20%) that can move freely throughout the study. The compound is administered intravenously and orally (gavage) in separate sets of animals. The applied doses for oral administration may range from 0.5 to 50 mg/kg body weight; the doses for intra-venous administration may range from 0.5 to 20 mg/kg body weight. Blood samples are collected through the catheter before compound administration and over the subsequent 24-hour period using an automated sampling device (AccuSampler, DiLab Europe, Lund, Sweden). Plasma levels of the compound are determined using a validated LC-MS analytical method. The pharmacokinetic analysis is performed on the plasma concentration-time curves after averaging all plasma concentrations across time points for each route of administration. Typical pharmacokinetics parameters to be calculated include: maximum concentration (Cₘₐₓ), time to maximum concentration (tₘₐₓ), area under the curve from 0 hours to the time point of the last quantifiable concentration (AUC₀₋ₜ), area under the curve from time 0 to infinity (AUC_{0-inf}), elimination rate constant (K), terminal half-life (t_{1/2}), absolute oral bioavailability or fraction absorbed (F), clearance (CL), and Volume of distribution during the terminal phase (Vd).

The compounds of the formula (I) and their pharmaceutically acceptable salts can be used as medicines, e.g. in the form of pharmaceutical compositions. The pharmaceutical compositions can be administered enterally, such as orally, e.g. in the form of tablets, lacquered tablets, sugar-coated tablets, hard and soft gelatine capsules, solutions, emulsions or suspensions, nasally, e.g. in the form of nasal sprays, rectally, e.g. in the form of suppositories, or transdermally, e.g. in the form of ointments or patches. However, administration is also possible parenterally, such as intramuscularly or intravenously, e.g. in the form of solutions for injection.

Tablets, lacquered tablets, sugar-coated tablets and hard gelatine capsules can be produced by processing the compounds of the formula (I) and their pharmaceutically acceptable salts with pharmaceutically inert inorganic or organic excipients. Excipients of these types which can be used for example for tablets, sugar-coated tablets and hard gelatine capsules are lactose, maize starch or derivatives thereof, talc, stearic acid or salts thereof etc.

Excipients suitable for soft gelatine capsules are, for example, vegetable oils, waxes, fats, semisolid and liquid polyols etc.

Excipients suitable for producing solutions and syrups are, for example, water, polyols, sucrose, invert sugar, glucose etc.

Excipients suitable for solutions for injection are, for example, water, alcohols, polyols, glycerol, vegetable oils, bile acids, lecithin etc.

Excipients suitable for suppositories are, for example, natural or hardened oils, waxes, fats, semiliquid or liquid polyols etc.

The pharmaceutical compositions may in addition comprise preservatives, solubilizers, viscosity-increasing substances, stabilizers, wetting agents, emulsifiers, sweeteners, colorants, aromatizers, salts to alter the osmotic pressure, buffers, coating agents or antioxidants. They may also comprise other substances of therapeutic value.

The present invention further provides the use of the compounds of the formula (I) and their pharmaceutically acceptable salts in the treatment or prevention of high blood pressure, heart failure, glaucoma, myocardial infarction, renal failure or restenoses.

The compounds of the formula (I) and their pharmaceutically acceptable salts can also be administered in combination with one or more agents having cardiovascular activity, e.g. alpha- and beta-blockers such as phentolamine, phenoxybenzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as amrinone, bencyclan, diltiazem, fendiline, flunarizine, nicardipine, nimodipine, perhexiline, verapamil, gallopamil, nifedipine etc.; ACE inhibitors such as cilazapril, captopril, enalapril, lisinopril etc.; potassium activators such as pinacidil; antiserotoninergics such as ketanserine; thromboxane synthetase inhibitors; neutral endopeptidase inhibitors (NEP inhibitors); angiotensin II antagonists; and diuretics such as hydrochlorothiazide, chlorothiazide, acetazolamide, amiloride, bumetanide, benzthiazide, ethacrynic acid, furosemide, indacrinone, metolazone, spironolactone, triamterene, chlorthalidone etc.; sympatholytics such as methyldopa, clonidine, guanabenz, reserpine; and other agents suitable for the treatment of high blood pressure, heart failure or vascular disorders associated with diabetes or renal disorders such as acute or chronic renal failure in humans and animals. Such combinations can be used separately or in products which comprise a plurality of components.

Further substances which can be used in combination with the compounds of formula (I) are the compounds of classes (i) to (ix) on page 1 of WO 02/40007 (and the preferences and examples detailed further therein) and the substances mentioned on pages 20 and 21 of WO 03/027091.

The dosage may vary within wide limits and must of course be adapted to the individual circumstances in each individual case. In general, a daily dose appropriate for oral administration ought to be from about 3 mg to about 3 g, preferably about 10 mg to about 1 g, e.g. approximately 300 mg per adult person (70 kg), divided into preferably 1-3 single doses, which may be for example of equal size, although the stated upper limit may also be exceeded if this proves to be indicated, and children usually receive a reduced dose appropriate for their age and body weight.

### EXAMPLES

The following examples illustrate the present invention. All temperatures are stated in degrees Celsius and pressures in mbar. Unless mentioned otherwise, the reactions take place at RT. The abbreviation “Rf=xx(A)” means for example that the Rf is found in solvent system A to be xx. The ratio of amounts of solvents to one another is always stated in parts by volume. Chemical names for final products and intermediates have been generated on the basis of the chemical structural formulae with the aid of the AutoNom 2000 (Automatic Nomenclature) program.

Thin-layer chromatography element systems:

A Dichloromethane/MeOH/conc. ammonia 25%=200:20:1

B Dichloromethane/MeOH/conc. ammonia 25%=200:20:0.5

C Dichloromethane/MeOH/conc. ammonia 25%=200:10:1

D Dichloromethane/MeOH/conc. ammonia 25%=90:10:1

E Dichloromethane/MeOH/conc. ammonia 25%=60:10:1

F Dichloromethane/MeOH/conc. ammonia 25%=200:30:1

G Dichloromethane/MeOH=9:1

H Dichloromethane/MeOH/conc. ammonia 25%=200:15:1

HPLC gradients on Hypersil BDS C-18 (5 um); column: 4×125 mm

90% water/10% acetonitrile* to 0% water*/100% acetonitrile* in 5 minutes+2.5 minutes (1.5 ml/min) (I)

95% water/5% acetonitrile* to 0% water*/100% acetonitrile* in 30 minutes+5 minutes (0.8 ml/min) (II)

* contains 0.1% trifluoroacetic acid

The following abbreviations are used:

AcOH acetic acid

BOC butyloxycarbonyl

n-BuLi n-butyllithium

t-BuOH tert-butanol

CH₂Cl₂ dichloromethane

CHCl₃ chloroform

CH₃CN acetonitrile

Cy cyclohexane

DCC dicyclohexylcarbodiimide

DIBAL diisobutylaluminium hydride

DMA dimethylacetamide

DME 1,2-dimethoxyethane

DMF N,N-dimethylformamide

EDC.HCl N-ethyl-N′-(3-dimethylaminopropyl)carbodiimide hydrochloride [25952-53-8]

Et₃N triethylamine

Et₂O diethylether

EtOAc ethyl acetate

EtOH ethanol

h hour(s)

HBr hydrobromic acid

HCl hydrochloric acid

H₂O water

K₂CO₃ potassium carbonate

KOH potassium hydroxide

LiCl lithiumchloride

MeI methyl iodide

MeOH methanol

min minute(s)

m.p. melting point (temperature)

N₂ nitrogen

NaBH₄ sodium borohydride

Na₂CO₃ sodium carbonate

NaH sodium hydride

NaHCO₃ sodium bicarbonate

NaOH sodium hydroxide

Na₂SO₄ sodium sulfate

NH₃ ammonia

NH₄Br ammonium bromide

NH₄Cl ammonium chloride

NH₄OH ammonium hydroxide

Pd₂(dba)₃ tris(dibenzylideneacetone)dipalladium [51364-51-3]

Pd(PPh₃)₄ tetrakis-triphenylphosphine palladium(0)

P(tert-Bu)₃ tri-tert-butylphosphine

Ra/Ni Raney-nickel

Rf ratio of distance which a substance travels to distance of the eluent front from the start point in thin layer chromatography

Rt retention time of a substance in HPLC (in minutes)

RT room temperature (23° C.)

TBAF tetrabutylammonium fluoride

TBAI tetrabutylammonium iodide

TBME tert-butyl methyl ether

TFA trifluoroacetic acid

THF tetrahydrofuran

General Method A: (N-BOC Deprotection)

To a solution of 1.0 mmol of “N-BOC derivative” in 10 mL of CH₂Cl₂ at 0° C. are added 20.0 mmol of TFA, and the reaction mixture is stirred at 0° C. for 1-5 h. The solution is quenched at 0° C. with ice-cold saturated aqueous NaHCO₃ solution, the mixture is stirred for 15 min, then extracted with TBME (2×). The combined organic extracts are washed with saturated aqueous NaHCO₃ solution, dried over Na₂SO₄ and concentrated under reduced pressure. The title compound is obtained from the residue by means of flash chromatography (SiO₂ 60F).

General Method B (Borane Reduction)

A solution of 1.0 mmol of “lactam” in 3 mL of THF is admixed with 3.0-6.0 mmol of borane-THF complex (1M in THF) and stirred at RT for 1-72 h (monitoring of conversion with HPLC or TLC). The reaction mixture is cooled to RT, admixed with MeOH (3.0-6.0 equiv.) and concentrated under reduced pressure. The title compound is obtained from the residue by means of flash chromatography (SiO₂ 60F).

General Method C: (Alcohol Alkylation)

A solution of 1.0 mmol of “alcohol”, 1.05 mmol of “halomethylaryl” and 1.1 mmol of TBAI in 4.0 mL of DMF is stirred at 0° C. for 10 min, before the addition of 1.1 mmol of NaH (60% dispersion in oil). The reaction mixture is stirred at 0° C. for 1 h, then at RT for 3-20 h, and poured into 1M aqueous NaHCO₃ solution. The mixture is extracted with CH₂Cl₂ (2×). The combined organic extracts are washed with brine, dried over Na₂SO₄ and concentrated under reduced pressure. The title compound is obtained from the residue by means of flash chromatography (SiO₂ 60F).

General Method D: (Phenol Alkylation)

A suspension of 1.0 mmol of “phenol”, 1.0-1.5 mmol of “tosylate” or “bromide”, 1.5 mmol of caesium carbonate and 2.0 mL of CH₃CN is stirred at 80° C. over 2 h. The reaction mixture is cooled, poured onto water and extracted with EtOAc (2×). The organic phases are washed with brine, dried over Na₂SO₄ and concentrated under reduced pressure. The title compound is obtained from the residue by means of flash chromatography (SiO₂ 60F).

### Example 361

### (3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-piperidin-4-ol

According to general method A, (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.19 (CH₂Cl₂/MeOH/conc. NH₃ 200:20:1); Rt=3.26 (gradient I).

The starting materials are prepared as follows:

### a) (3S,4S)-4-Hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

According to general method B, (3S,4S)-4-hydroxy-4-[4-(2-methoxy-ethoxymethyl)-phenyl]-3-[4-(3-methoxypropyl)-3-oxo-3,4-dihydro-2H-benzo[1,4]oxazin-6-ylmethoxy]-piperidine-1-carboxylic acid tert-butyl ester is used to afford the title compound as a yellow oil. Rf=0.31 (EtOAc/heptane 1:1); Rt=4.80 (gradient I).

### b) (3S,4S)-4-Hydroxy-4-[4-(2-methoxy-ethoxymethyl)-phenyl]-3-[4-(3-methoxy-propyl)-3-oxo-3,4-dihydro-2H-benzo[1,4]oxazin-6-ylmethoxy]-piperidine-1-carboxylic acid tert-butyl ester

According to general method C, (3S,4S)-3,4-dihydroxy-4-[4-(2-methoxy-ethoxy-methyl)-phenyl]-piperidine-1-carboxylic acid tert-butyl ester and 7.1 mmol of 6-bromomethyl-4-(3-methoxy-propyl)-4H-benzo[1,4]oxazin-3-one [91170542-5] is used to afford the title compound as a yellow oil. Rf=0.32 (EtOAc/heptane 2:1); Rt=4.48 (gradient I).

### c) (3S,4S)-3,4-Dihydroxy-4-[4-(2-methoxy-ethoxymethyl)-phenyl]-piperidine-1-carboxylic acid tert-butyl ester

To a stirred solution of (38.3 g) of AD-mix-a [ALDRICH, 39,275-8, lot 01614BE/277] in 80 mL of t-BuOH and 80 mL of H₂O are added 22.4 mmol of methansulfonamide. The reaction mixture is cooled to 0° C., followed by the addition of 22.4 mmol of 4-[4-(2-methoxy-ethoxymethyl)-phenyl]-3,6-dihydro-2H-pyridine-1-carboxylic acid tert-butyl ester in 35 mL of t-BuOH and 35 mL of H₂O. The reaction mixture is stirred at 0° C. for 30 min, then at RT for 3 days. To the reaction mixture is added 33 g of Na₂SO₃ followed by stirring for 1 h. CH₂Cl₂ (250 mL) is added, the layers are separated and the aqueous layer is extracted again with CH₂Cl₂ (4×150 mL). The combined organic layers are washed with 2N aqueous KOH (200 mL), dried over Na₂SO₄ and concentrated under reduced pressure. The residue is purified by flash chromatography (SiO₂ 60F) to afford the title compound as a yellow oil. Rf=0.06 (EtOAc/heptane 1:2); Rt=3.52 (gradient I).

### d) 4-[4-(2-Methoxy-ethoxymethyl)-phenyl]-3,6-dihydro-2H-pyridine-1-carboxylic acid tert-butyl ester

A three-neck flask is charged with 22.2 mmol of 4-trifluoromethane-sulfonyloxy-3,6-dihydro-2H-pyridine-1-carboxylic acid tert-butyl ester [138647-49-1], 30.2 mmol of 4-(2-methoxy-ethoxymethyl)-phenylboronic acid, 66.7 mmol of LiCl, 105 mL of 2N aqueous Na₂CO₃ solution, 220 mL of DME and 1.1 mmol of Pd(PPh₃)₄. The reaction is heated to reflux for 3 h followed by cooling to RT and concentration under reduced pressure. The residue is partitioned between CH₂Cl₂ (500 mL), 2N aqueous Na₂CO₃ solution (400 mL) and concentrated NH₄OH solution (25 mL). The layers are separated and the aqueous layer is extracted again with CH₂Cl₂ (3×500 mL). The combined organic layers are dried over Na₂SO₄ and concentrated under reduced pressure. The resulting black residue is purified by flash chromatography (SiO₂ 60F) to afford of the title compound as a yellow oil. Rf=0.50 (EtOAc/heptane 1:1); Rt=4.81 (gradient I).

### e) 4-(2-Methoxy-ethoxymethyl)-phenylboronic acid

A solution of 38.8 mmol of n-BuLi (1.6 M in hexanes) is added dropwise to a stirred solution of 32.3 mmol of 1-bromo-4-(2-methoxy-ethoxymethyl)-benzene [166959-29-1] in 50 mL of THF at −78° C. The reaction mixture is stirred for 30 min at −78° C. and 64.6 mmol of triisopropyl borate are added rapidly. The mixture is stirred for 30 min at −78° C. and at RT for 1 h. The reaction mixture is partitioned between 2N aqueous HCl solution (40 mL) and EtOAc (300 mL). The organic layer is washed with brine (2×50 mL), dried over Na₂SO₄ and concentrated under reduced pressure to afford the title compound as a yellow oil. Rt=2.52 (gradient I).

### Example 367

### (3S,4S)-4-[4-((S)-3-Methoxy-2-methyl-propoxymethyl)-phenyl]-3-[4-(3-methoxy-propyl)-3,4-dihydro-2H-benzo[1,4]oxazin-6-ylmethoxy]-piperidin-4-ol

According to general method A, (3S,4S)-4-hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.24 (CH₂Cl₂/MeOH/conc. NH₃ 200:20:1); Rt=3.84 (gradient I).

The starting materials are prepared as follows:

### a) (3S,4S)-4-Hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

According to the processes described in example 361 (a, b, c, d) and starting from 4-((R)-3-methoxy-2-methyl-propan-1-oxymethyl)-phenylboronic, the title compound is obtained as a yellow oil. Rf=0.28 (EtOAc/heptane 1:1); Rt=5.56 (gradient I).

### b) 4-((R)-3-Methoxy-2-methyl-propan-1-oxymethyl)-phenylboronic acid

According to the process described in example 361e, 1-bromo-4-((S)-3-methoxy-2-methyl-propoxymethyl)-benzene is used to afford the title compound as a yellow oil. Rt=3.36 (gradient I).

### c) 1-Bromo-4-((S)-3-methoxy-2-methyl-propoxymethyl)-benzene

A solution of 501.9 mmol of (R)-3-methoxy-2-methyl-propan-1-ol [911855-78-2] in 100 mL of DMF is added for 30 min to an ice-cooled suspension of 602.2 mmol of NaH (60% dispersion in oil) in 250 mL of DMF. The suspension is stirred at 0° C. for 30 min then a solution of 401.5 mmol of 1-bromo-4-chloromethyl-benzene in 100 mL of THF is added over 30 min. The reaction mixture is stirred at RT for 4 h, diluted with TBME (750 mL) and washed with aqueous saturated NaHCO₃ solution (750 mL). The aqueous phase is extracted with TBME (3×1 L). The combined organic layers are washed successively with water (350 mL) and brine (350 mL), dried over Na₂SO₄ and concentrated under reduced pressure. The residue is purified by flash chromatography (SiO₂ 60F), to afford the title compound as a yellowish oil. Rf=0.43; Rt=5.28 (gradient I).

### Example 369

### (3S,4S)-4-[4-((R)-2-Ethoxy-propoxymethyl)-phenyl]-3-[4-(3-methoxy-propyl)-3,4-dihydro-2H-benzo[1,4]oxazin-6-ylmethoxy]-piperidin-4-ol

According to general method A, (3S,4S)-4-[4-((R)-2-ethoxy-propoxymethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellowish wax. Rf=0.13 (CH₂Cl₂/MeOH/conc NH₃ 200:20:1); Rt=3.74 (gradient I).

The starting materials are prepared as follows:

### a) (3S,4S)-4-[4-((R)-2-Ethoxy-propoxymethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

Analogously to the processes described in example 361 (a-d) and starting from [4-((R)-2-ethoxy-propoxymethyl)-phenyl]-dimethyl-boronic acid, the title compound is obtained as a yellowish solid. Rf=0.21 (EtOAc/heptane 1:1); Rt=5.48 (gradient I).

### b) [4-((R)-2-Ethoxy-propoxymethyl)-phenyl]-dimethyl-boronic acid

Analogously to the process described in example 361 (e), 1-bromo-4-((R)-2-ethoxy-propoxymethyl)-benzene is used to afford the title compound as a yellow greenish oil. Rt=3.16 (gradient I).

### c) 1-Bromo-4-((R)-2-ethoxy-propoxymethyl)-benzene

To a solution of 61.60 mmol of (R)-1-(4-bromo-benzyloxy)-propan-2-ol in 115 mL of DMF are added 101.65 mmol of NaH (55% dispersion in oil). The reaction mixture is stirred for 1 h at RT then 110.89 mmol of ethyliodide are added over 5 minutes. The reaction mixture is stirred at RT for 18 h, then poured into saturated aqueous NH₄Cl solution (200 mL) and extracted with TBME (2×250 mL). The combined organic layers are washed successively with H₂O (2×100 mL) and brine (1×100 mL), dried over Na₂SO₄ and concentrated under reduced pressure. The residue is purified by flash chromatography (SiO₂ 60F) to afford the title compound as a yellow oil. Rf=0.63 (EtOAc/heptane 1:2); Rt=4.85 (gradient I).

### d) (R)-1-(4-Bromo-benzyloxy)-propan-2-ol

A solution containing 66.23 mmol of (R)-2-(4-bromo-benzyloxymethyl)-oxirane and 397.36 mmol of NaBH₄ in 165 mL of EtOH and 16.5 mL of THF is stirred at 55° C. for 3 h, cooled to RT then poured into 700 mL of cold 1N NH₄Cl solution. The mixture is extracted with TBME (2×700 mL). The combined organic layers are washed with brine (700 mL), dried over Na₂SO₄, filtered and concentrated under reduced pressure. The residue is purified by flash chromatography (SiO₂ 60F) to afford the title compound as a colourless oil. Rf=0.50 (EtOAc/heptane 1:1); Rt=3.77 (gradient I).

### Example 370

### (3S,4S)-3-[4-(3-Methoxy-propyl)-3,4-dihydro-2H-benzo[1,4]oxazin-6-ylmethoxy]-4-[4-((S)-2-methyl-3-tetrazol-2-yl-propoxymethyl)-phenyl]-piperidin-4-ol

According to general method A, (3S,4S)-4-hydroxy-3-[4-(3-methoxy-propyl)-3,4-dihydro-2H-benzo[1,4]oxazin-6-ylmethoxy]-4-[4-((S)-2-methyl-3-tetrazol-2-yl-propoxy-methyl)-phenyl]-piperidine-1-carboxylic acid tert-butyl ester is used to afford the title compound as a yellow wax. Rf=0.15 (EtOAc/heptane 1:1); Rt=5.22 (gradient I).

The starting materials are prepared as follows:

### a) 3S,4S)-4-Hydroxy-3-[4-(3-methoxy-propyl)-3,4-dihydro-2H-benzo[1,4]oxazin-6-ylmethoxy]-4-[4-((S)-2-methyl-3-tetrazol-2-yl-propoxymethyl)-phenyl]-piperidine-1-carboxylic acid tert-butyl ester

To a solution of 8.88 mmol of 1H-tetrazole in 50 mL of DMF are added 7.10 mmol of NaH (60% dispersion in oil). The mixture is stirred at RT for 45 min, then warmed to 65° C. At this temperature, a solution of 2.54 mmol of (3S,4S)-4-hydroxy-3-[4-(3-methoxy-propyl)-3,4-dihydro-2H-benzo[1,4]oxazin-6-ylmethoxy]-4-{4-[(R)-2-methyl-3-(toluene-4-sulfonyloxy)-propoxymethyl]-phenyl}-piperidine-1-carboxylic acid tert-butyl ester in 20 mL of DMF is added over 5 min. The reaction mixture is stirred for 16 h at 65° C., cooled to RT and poured into water. The mixture is extracted twice with TBME. The combined organic phases are washed with brine, dried over Na₂SO₄, filtered and concentrated under reduced pressure. The residue is purified by flash chromatography (SiO₂ 60F) to afford the title compound as a yellowish solid. Rf=0.33 (EtOAc/heptane 2:1); Rt=5.23 (gradient I).

### b) (3S,4S)-4-Hydroxy-3-[4-(3-methoxy-propyl)-3,4-dihydro-2H-benzo[1,4]oxazin-6-ylmethoxy]-4-{4-[(R)-2-methyl-3-(toluene-4-sulfonyloxy)-propoxymethyl]-phenyl}-piperidine-1-carboxylic acid tert-butyl ester

To a solution of 3.29 mmol of (3S,4S)-4-hydroxy-4-[4-((S)-3-hydroxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester and 9.88 mmol of Et₃N in 70 mL of CH₂Cl₂ are added 5.44 mmol of p-toluenesulfonyl chlorie, followed by 0.329 mmol of 4-dimethylaminopyridine. The reaction mixture is stirred at RT for 16 h then poured into aqueous 1N NaHCO₃ solution. The aqueous phase is extracted twice with CH₂Cl₂. The combined organic phases are washed with brine, dried over Na₂SO₄, filtered and concentrated under reduced pressure. The residue is purified by flash chromatography (SiO₂ 60F) to afford the title compound as a yellowish oil. Rf=0.20 (EtOAc/heptane 1:1); Rt=5.80 (gradient I).

### c) (3S,4S)-4-Hydroxy-4-[4-((S)-3-hydroxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

To an ice cold solution of 0.794 mmol of (3S,4S)-4-hydroxy-3-[4-(3-methoxy-propyl)-3,4-dihydro-2H-benzo[1,4]oxazin-6-ylmethoxy]-4-[4-((R)-2-methyl-3-triisopropyl-silanyloxy-propoxymethyl)-phenyl]-piperidine-1-carboxylic acid tert-butyl ester in 12 mL of THF is added dropwise 2.382 mmol of TBAF (1 N in THF). The reaction mixture is stirred at 0° C. for 2 h, poured into water and extracted twice with CH₂Cl₂. The combined organic phases are washed with brine, dried over Na₂SO₄, filtered and concentrated under reduced pressure. The title compound is obtained as a colourless solid from the residue. Rf=0.19 (EtOAc/heptane 2:1); Rt=4.81 (gradient I).

### d) (3S,4S)-4-Hydroxy-3-[4-(3-methoxy-propyl)-3,4-dihydro-2H-benzo[1,4]oxazin-6-ylmethoxy]-4-[4-((R)-2-methyl-3-triisopropylsilanyloxy-propoxymethyl)-phenyl]-piperidine-1-carboxylic acid tert-butyl ester

Analogously to the processes described for example 361 (a-d) and starting from 4-((R)-2-methyl-3-triisopropylsilanyloxy-propoxymethyl)-phenyl-boronic acid, the title compound is obtained as a yellow oil. Rf=0.21 (EtOAc/heptane 1:10).

### e) 4-((R)-2-Methyl-3-triisopropylsilanyloxy-propoxymethyl)-phenyl-boronic acid

Analogously to the process described for example 361 (e), [(R)-3-(4-bromo-benzyloxy)-2-methyl-propoxy]-triisopropyl-silane is used to obtain the title compound as a colourless oil. Rt=6.28 (gradient I).

### f) [(R)-3-(4-Bromo-benzyloxy)-2-methyl-propoxy]-triisopropyl-silane

To an ice cold solution of 55.74 mmol of (S)-3-(4-bromo-benzyloxy)-2-methyl-propan-1-ol and 61.32 mmol of imidazole in 100 mL of CH₂Cl₂ are added 58.53 mmol of triisopropylchlorosilane. The reaction mixture is stirred at RT for 15 h, diluted with CH₂Cl₂ (400 mL), washed sequentially with aqueous 0.1N HCl solution (100 mL), water (100 mL) and brine (100 mL), dried over Na₂SO₄, filtered and concentrated under reduced pressure. The residue is purified by flash chromatography (SiO₂ 60F) to afford the title compound as a colourless oil. Rt=7.50 (gradient I).

### g) (S)-3-(4-Bromo-benzyloxy)-2-methyl-propan-1-ol

To an ice cold solution of 797 mmol of (R)-3-(4-bromo-benzyloxy)-2-methyl-propionic acid methyl ester in 1500 mL of TBME are added 1203 mmol of lithium borohydride portionwise over 45 min. The turbid reaction mixture is stirred at 0° C. for 1 h, then at RT for 24 h, and diluted with 200 mL of TBME. After cooling at 0° C., 1500 mL of aqueous saturated NH₄Cl solution are carefully added. The biphasic mixture is vigorously stirred at RT for 2 h. The aqueous phase is separated and extracted with 1000 mL of TBME. The combined organic phases are washed with water (1000 mL) then brine (500 mL), dried over Na₂SO₄, filtered and concentrated under reduced pressure. The title compound is obtained as a yellowish oil from the residue. Rf=0.27 (EtOAc/heptane 1:2); Rt=4.20 (gradient I).

### h) (R)-3-(4-Bromo-benzyloxy)-2-methyl-propionic acid methyl ester

To an ice cold solution of 1508 mmol of 2,2,2-trichloro-acetimidic acid 4-bromo-benzyl ester [146285-52-1] and 1257 mmol of (R)-(−)-3-hydroxy-2-methylpropionic acid methyl ester in 600 mL CH₂Cl₂ and 800 mL of cyclohexane are added dropwise 0.075 mmol of trifluoromethanesulfonic acid over 15 min. The reaction mixture is vigorously stirred for 70 min at 0° C., diluted with CH₂Cl₂ (1500 mL) and washed with aqueous saturated NaHCO₃ solution (1000 mL). The aqueous phase is extracted with CH₂Cl₂ (2×500 mL). The combined organic phases are washed with brine (250 mL), dried over Na₂SO₄, filtered and evaporated. The residue is purified by flash chromatography (SiO₂ 60F) to afford the title compound as a yellowish oil. Rf=0.22 (EtOAc/heptane 1:9); Rt=4.72 (gradient I).

### Example 371

### (3S,4S)-3-[4-(3-Methoxy-propyl)-3,4-dihydro-2H-benzo[1,4]oxazin-6-ylmethoxy]-4-[4-((S)-2-methyl-3-tetrazol-1-yl-propoxymethyl)-phenyl]-piperidin-4-ol

According to general procedure A, (3S,4S)-4-hydroxy-3-[4-(3-methoxy-propyl)-3,4-dihydro-2H-benzo[1,4]oxazin-6-ylmethoxy]-4-[4-((S)-2-methyl-3-tetrazol-1-yl-propoxy-methyl)-phenyl]-piperidine-1-carboxylic acid tert-butyl ester is used to afford the title compound as a yellow oil. Rf=0.13 (EtOAc/heptane 2:1); Rt=4.92 (gradient I).

The starting materials are prepared as follows:

### a) (3S,4S)-4-Hydroxy-3-[4-(3-methoxy-propyl)-3,4-dihydro-2H-benzo[1,4]oxazin-6-ylmethoxy]-4-[4-((S)-2-methyl-3-tetrazol-1-yl-propoxymethyl)-phenyl]-piperidine-1-carboxylic acid tert-butyl ester

To a solution of 8.88 mmol of 1H-tetrazole in 50 mL of DMF are added 7.10 mmol of NaH (60% dispersion in oil). The mixture is stirred at RT for 45 min then warmed to 65° C. At this temperature, a solution of 2.54 mmol of (3S,4S)-4-hydroxy-3-[4-(3-methoxy-propyl)-3,4-dihydro-2H-benzo[1,4]oxazin-6-ylmethoxy]-4-{4-[(R)-2-methyl-3-(toluene-4-sulfonyloxy)-propoxymethyl]-phenyl}-piperidine-1-carboxylic acid tert-butyl ester (example 371 b) in 20 mL of DMF is added over 5 min. The reaction mixture is stirred for 16 h at 65° C., cooled to RT and poured into water. The mixture is extracted twice with TBME. The combined organic phases are washed with brine, dried over Na₂SO₄, filtered and concentrated under reduced pressure. The residue is purified by flash chromatography (SiO₂ 60F) to afford the title compound as a yellowish solid. Rf=0.13 (EtOAc/heptane 2:1); Rt=4.92 (gradient I).

### Example 372

### (3S,4S)-3-[4-(3-Methoxy-propyl)-3,4-dihydro-2H-benzo[1,4]oxazin-6-ylmethoxy]-4-{4-[(S)-1-(2-methyl-2H-tetrazol-5-yl)-pyrrolidin-3-yloxy]-phenyl}-piperidin-4-ol

According to general method A, (3S,4S)-4-hydroxy-3-[4-(3-methoxy-propyl)-3,4-dihydro-2H-benzo[1,4]oxazin-6-ylmethoxy]-4-{4-[(S)-1-(2-methyl-2H-tetrazol-5-yl)-pyrrolidin-3-yloxy]-phenyl}-piperidine-1-carboxylic acid tert-butyl ester is used to afford the title compound as a yellow oil. Rf=0.05 (EtOAc/heptane 1:1); Rt=4.98 (gradient I).

The starting materials are prepared as follows:

### a) (3S,4S)-4-Hydroxy-3-[4-(3-methoxy-propyl)-3,4-dihydro-2H-benzo[1,4]oxazin-6-ylmethoxy]-4-{4-[(S)-1-(2-methyl-2H-tetrazol-5-yl)-pyrrolidin-3-yloxy]-phenyl}-piperidine-1-carboxylic acid tert-butyl ester

According to general method D, (3S,4S)-4-hydroxy-4-(4-hydroxy-phenyl)-3-[4-(3-methoxy-propyl)-3,4-dihydro-2H-benzo[1,4]oxazin-6-ylmethoxy]-piperidine-1-carboxylic acid tert-butyl ester and 0.754 mmol of toluene-4-sulfonic acid (R)-1-(2-methyl-2H-tetrazol-5-yl)-pyrrolidin-3-yl ester is used to obtain the title compound as a yellow oil. Rf=0.05 (EtOAc/heptane 1:1); Rt=4.98 (gradient I).

### b) (3S,4S)-4-Hydroxy-4-(4-hydroxy-phenyl)-3-[4-(3-methoxy-propyl)-3,4-dihydro-2H-benzo[1,4]oxazin-6-ylmethoxy]-piperidine-1-carboxylic acid tert-butyl ester

To a solution of 0.954 mmol of (3S,4S)-4-[4-(tert-butyl-dimethyl-silanyloxy)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester in 10 mL of THF are added dropwise 1.908 mmol of TBAF (1 N in THF). The reaction mixture is stirred at RT for 30 min, poured into water (25 mL) and extracted with TBME (3×20 mL). The combined organic phases are washed with brine (20 mL), dried over Na₂SO₄, filtered and concentrated under reduced pressure. The residue is purified by flash chromatography (SiO₂ 60F) to afford the title compound as a white turbid oil. Rf=0.32 (EtOAc/heptane 1:1); Rt=4.54 (gradient I).

### c) (3S,4S)-4-[4-(tert-Butyl-dimethyl-silanyloxy)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

Analogously to the processes described in example 361 (a-b) and starting from (3S,4S)-4-[4-(tert-butyl-dimethyl-silanyloxy)-phenyl]-3,4-dihydroxy-piperidine-1-carboxylic acid tert-butyl ester [357608-36-7], the title compound is obtained as a yellow oil. Rf=0.61 (EtOAc/heptane 1:1); Rt=6.28 (gradient I).

### d) Toluene-4-sulfonic acid (R)-1-(2-methyl-2H-tetrazol-5-yl)-pyrrolidin-3-yl ester

Analogously to the process described in example 370b, (R)-1-(2-methyl-2H-tetrazol-5-yl)-pyrrolidin-3-ol is used to obtain the title compound as white crystals. Rf=0.70 (CH₂Cl₂/MeOH 20:1); Rt=3.88 (gradient I).

### e) (R)-1-(2-Methyl-2H-tetrazol-5-yl)-pyrrolidin-3-ol

To a stirred suspension of 11.60 mmol of (R)-1-(2H-tetrazol-5-yl)-pyrrolidin-3-ol and 13.92 mmol of Na₂CO₃ in 50 mL of DMF are added 15.08 mmol of ethyliodide. The reaction mixture is stirred at RT for 4 h, filtered and concentrated under reduced pressure. The residue is purified by flash chromatography (SiO₂ 60F) to afford the title compound as white crystals. Rf=0.57 (EtOAc/MeOH 10:1).

### f) (R)-1-(2H-Tetrazol-5-yl)-pyrrolidin-3-ol

To a solution of 23.74 mmol of (R)-1-(benzotriazol-1-yl-imino-methyl)-pyrrolidin-3-ol in 85 mL of CHCl₃ are added 23.74 mmol of sodium azide, followed by 23.74 mmol of AcOH. The reaction mixture is stirred at RT for 15 h then concentrated under reduced pressure. The residue is purified by flash chromatography (SiO₂ 60F) to afford the title compound as white crystals. Rf=0.15 (EtOAc/MeOH 10:1).

### g) (R)-1-(Benzotriazol-1-yl-imino-methyl)-pyrrolidin-3-ol

A solution of 33.43 mmol of (R)-(+)-3-pyrrolininol in 30 mL of CH₂Cl₂ is added to a solution of 33.43 mmol of C-(bis-benzotriazol-1-yl)-methylene amine [28992-50-9] in 180 mL of CH₂Cl₂. The reaction mixture is stirred at RT for 15 h, washed with aqueous 10% NaHCO₃ (2×75 mL), dried over Na₂SO₄, filtered and evaporated to afford the title compound as an orange oil. LC-MS: 232 (M+H).

### Example 373

### (3S,4S)-4-[4-((R)-2-Ethoxy-propoxymethyl)-2-methoxy-phenyl]-3-[4-(3-methoxy-propyl)-3,4-dihydro-2H-benzo[1,4]oxazin-6-ylmethoxy]-piperidin-4-ol

According to general method A, (3S,4S)-4-[4-((R)-2-ethoxy-propoxymethyl)-2-methoxy-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester is used to afford the title compound as a light yellow oil. Rf=0.49 (CH₂Cl₂/MeOH/conc. NH₃ 40:10:1); Rt=3.93 (gradient I).

The starting materials are prepared as follows:

### a) (3S,4S)-4-[4-((R)-2-Ethoxy-propoxymethyl)-2-methoxy-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

According to the processes described in example 361 (a, b, c, d) and starting from 4-((R)-2-ethoxy-propoxymethyl)-2-methoxy-phenyl-boronic acid, the title compound is obtained as a light yellow oil. Rf=0.16 (EtOAc/heptane 1:1); Rt=5.60 (gradient I).

### b) 4-((R)-2-Ethoxy-propoxymethyl)-2-methoxy-phenyl-boronic acid

According to the process described in example 361e, 1-bromo-4-((R)-2-ethoxy-propoxymethyl)-2-methoxy-benzene is used to afford the title compound as a yellow oil. Rf=0.14 (EtOAc/heptane 1:2); Rt=3.57 (gradient I).

### c) 1-Bromo-4-((R)-2-ethoxy-propoxymethyl)-2-methoxy-benzene

To a solution of 16.14 mmol of 1-bromo-4-chloromethyl-2-methoxy-benzene [113081-49-5] and 20.98 mmol of (R)-2-ethoxy-propan-1-ol in 50 mL of DMF at −10° C. are added 19.36 mmol of NaH (55% suspension in oil), and 1.61 mmol of TBAI. The reaction mixture is stirred for 18 h, letting the temperature rise slowly to RT, then poured into 1M NaHCO₃ solution, and extracted with TBME (3×). The combined organic extracts are washed sequentially with water (2×) and brine, dried over Na₂SO₄, filtered and concentrated under reduced pressure. The residue is purified by flash chromatography (SiO₂ 60F) to afford the title compound as a light yellow oil. Rf=0.39 (EtOAc/heptane 1:2); Rt=4.95 (gradient I).

### d) (R)-2-Ethoxy-propan-1-ol

To a solution of 40.26 mmol of (R)-2-ethoxy-propionic acid methyl ester in 120 mL of Et₂O at 0° C. are added portionwise 62.40 mmol of lithium borohydride. The reaction mixture is stirred for 20 h, letting the temperature rise slowly to RT. The mixture is poured into 100 mL of ice-cold saturated NH₄Cl solution, and stirred for 10 min at 0° C., then for 30 min at RT. The phases are separated, the aqueous phase is extracted again 2× with Et₂O. The combined organic extracts are washed with brine (2×), dried over Na₂SO₄ and concentrated under reduced pressure (40° C., 500 mbar), to afford the title compound as a pale yellow oil. Rf=0.27 (CH₂Cl₂/Et₂O 3:1).

### d) (R)-2-Ethoxy-propionic acid methyl ester

To a solution of 68.16 mmol of methyl (R)-(+)-lactate [17392-83-5], 136.3 mmol of ethyl iodide in 60 mL of Et₂O, shielded from the light, are added 136.3 mmol of silver oxide. The reaction mixture is stirred at RT for 24 h. Additional ethyl iodide (68.2 mmol) and silver oxide (68.2 mmol) are added, and the reaction mixture is stirred at RT for another 24 h. The reaction mixture is filtered through hyflo, the filter cake is washed with CH₂Cl₂ and the filtrate is concentrated under reduced pressure (40° C., 500 mbar). The residue is purified by flash chromatography (SiO₂ 60F) to afford the title compound as a pale yellow oil. Rf=0.20 (pentane/Et₂O 9:1).

### Example 374

### (3S,4S)-4-[2-Ethoxy-4-((R)-2-ethoxy-propoxymethyl)-phenyl]-3-[4-(3-methoxy-propyl)-3,4-dihydro-2H-benzo[1,4]oxazin-6-ylmethoxy]-piperidin-4-ol

According to general method A, (3S,4S)-4-[2-ethoxy-4-((R)-2-ethoxy-propoxymethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester is used to afford the title compound as a light yellow oil. Rf=0.37 (CH₂Cl₂/MeOH/conc. NH₃ 90:10:1); Rt=3.93 (gradient I).

The starting materials are prepared as follows:

### a) (3S,4S)-4-[2-Ethoxy-4-((R)-2-ethoxy-propoxymethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

According to the processes described in example 361 (a, b, c, d) and starting from 2-ethoxy-4-((R)-2-ethoxy-propoxymethyl)-phenyl-boronic acid, the title compound is obtained as a light yellow oil. Rf=0.48 (EtOAc/heptane 2:1); Rt=5.76 (gradient I).

### b) 2-Ethoxy-4-((R)-2-ethoxy-propoxymethyl)-phenyl-boronic acid

According to the process described in example 361e, 1-bromo-2-ethoxy-4-((R)-2-ethoxy-propoxymethyl)-benzene is used to afford the title compound as a yellow oil. Rf=0.11 (EtOAc/heptane 1:2); Rt=3.91 (gradient I).

### c) 1-Bromo-2-ethoxy-4-((R)-2-ethoxy-propoxymethyl)-benzene

According to the process described in example 373c, 1-bromo-4-chloromethyl-2-ethoxy-benzene is used to afford the title compound as a pale yellow oil. Rf=0.47 (EtOAc/heptane 1:2); Rt=5.30 (gradient I).

### d) 1-Bromo-4-chloromethyl-2-ethoxy-benzene

To a solution of 36.20 mmol of (4-bromo-3-ethoxy-phenyl)-methanol in 80 mL of CH₂Cl₂ at 0° C. are added 44.17 mmol of Et₃N, followed by 3.62 mmol of tetrabutyl-ammonium chloride, and 40.55 mmol of methanesulfonyl chloride. The reaction mixture is stirred for 20 h, letting the temperature rise slowly to RT. The reaction mixture is poured into 1M NaHCO₃ solution, and extracted with CH₂Cl₂ (2×). The combined organic extracts are washed with brine, dried over Na₂SO₄ and concentrated under reduced pressure. The residue is purified by flash chromatography (SiO₂ 60F) to afford the title compound as a yellow oil. Rf=0.77 (EtOAc/heptane 1:1); Rt=5.11 (gradient I).

### e) (4-Bromo-3-ethoxy-phenyl)-methanol

To a solution of 65.79 mmol of 4-bromo-3-ethoxy-benzoic acid ethyl ester [220380-11-0] in 720 mL of THF are added portionwise 197.38 mmol of lithium borohydride. The reaction mixture is heated at 50° C., and stirred for 20 h. The mixture is cooled at RT, and additional lithium borohydride (197.38 mmol) is added. The reaction mixture is stirred at 70° C. for 24 h, cooled at RT, and more lithium borohydride (197.38 mmol) is added. The reaction mixture is stirred for 24 h at 70° C., cooled at RT and poured into 1 L of ice-cold saturated NH₄Cl solution, and stirred for 1 h at RT. The phases are separated, the aqueous phase is extracted again with TBME (2×). The combined organic extracts are washed with brine (2×), dried over Na₂SO₄ and concentrated under reduced pressure. The residue is purified by flash chromatography to afford the title compound as a yellow oil. Rf=0.40 (CH₂Cl₂/MeOH/conc. NH₃ 200:5:0.5); Rt=3.80 (gradient I).

### Example 375

### (3S,4S)-4-[4-((R)-2-Ethoxy-propoxymethyl)-2-(2-methoxy-ethoxy)-phenyl]-3-[4-(3-methoxy-propyl)-3,4-dihydro-2H-benzo[1.4]oxazin-6-ylmethoxy]-piperidin-4-ol

According to general method A, (3S,4S)-4-[4-((R)-2-ethoxy-propoxymethyl)-2-(2-methoxy-ethoxy)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester is used to afford the title compound as a light yellow oil. Rf=0.25 (CH₂Cl₂/MeOH/conc. NH₃ 90:10:1); Rt=3.87 (gradient I).

The starting materials are prepared as follows:

### a) (3S,4S)-4-[4-((R)-2-Ethoxy-propoxymethyl)-2-(2-methoxy-ethoxy)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

According to the processes described in example 361 (a, b, c, d) and starting from 4-((R)-2-ethoxy-propoxymethyl)-2-(2-methoxy-ethoxy)-phenyl-boronic acid, the title compound is obtained as a colourless oil. Rf=0.21 (EtOAc/heptane 1:1); Rt=5.53 (gradient I).

### b) 4-((R)-2-Ethoxy-propoxymethyl)-2-(2-methoxy-ethoxy)-phenyl-boronic acid

According to the process described in example 361e, 1-bromo-4-((R)-2-ethoxy-propoxymethyl)-2-(2-methoxy-ethoxy)-benzene are used to afford the title compound as a yellow oil. Rf=0.09 (EtOAc/heptane 1:2); Rt=3.62 (gradient I).

### c) 1-Bromo-4-((R)-2-ethoxy-propoxymethyl)-2-(2-methoxy-ethoxy)-benzene

According to the process described in example 373c, of 1-bromo-4-chloromethyl-2-(2-methoxy-ethoxy)-benzene is used to afford the title compound as a pale yellow oil. Rf=0.31 (EtOAc/heptane 1:2); Rt=4.84 (gradient I).

### d) 1-Bromo-4-chloromethyl-2-(2-methoxy-ethoxy)-benzene

Analogously to the process described in example 374d, [4-bromo-3-(2-methoxy-ethoxy)-phenyl]-methanol is used to afford the title compound as a yellowish green oil. Rf=0.41 (EtOAc/heptane 1:2); Rt=4.68 (gradient I).

### e) [4-Bromo-3-(2-methoxy-ethoxy)-phenyl]-methanol

Analogously to the process described in example 374e, 4-bromo-3-(2-methoxy-ethoxy)-benzoic acid methyl ester is used to afford the title compound as a colourless oil. Rf=0.27 (EtOAc/heptane 3:1); Rt=3.40 (gradient I).

### f) 4-Bromo-3-(2-methoxy-ethoxy)-benzoic acid methyl ester

To a suspension of 59.38 mmol of dry K₂CO₃, methyl 4-bromo-3-hydroxybenzoate [106291-80-9] and 2.12 mmol of TBAI in 200 mL of acetone are added dropwise 46.66 mmol of 2-bromoethyl methyl ether. The reaction mixture is stirred at 50° C. for 20 h. Additional 2-bromoethyl methyl ether (46.66 mmol) are added, and the reaction mixture is stirred at 75° C. for 3 h. Another portion of 2-bromoethyl methyl ether (46.66 mmol) is added, and the reaction mixture is stirred at 75° C. for 3 h. After adding another portion (46.66 mmol) of 2-bromoethyl methyl ether, the reaction mixture is stirred at 75° C. for 20 h, cooled to RT and filtered, washing the filter cake with acetone. The filtrate is concentrated under reduced pressure to afford the title compound as a pale yellow oil. Rf=0.28 (EtOAc/heptane 1:3); Rt=4.45 (gradient I).

### Example 376

### (3S,4S)-4-[4-((R)-2-Ethoxy-propoxymethyl)-2-methoxymethyl-phenyl]-3-[4-(3-methoxy-propyl)-3,4-dihydro-2H-benzo[1,4]oxazin-6-ylmethoxy]-piperidin-4-ol

According to general method A, (3S,4S)-4-[4-((R)-2-ethoxy-propoxymethyl)-2-methoxymethyl-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester is used to afford the title compound as an orange oil. Rf=0.25 (CH₂Cl₂/MeOH/conc. NH₃ 200:20:1); Rt=3.92 (gradient I).

The starting materials are prepared as follows:

### a) (3S,4S)-4-[4-((R)-2-Ethoxy-propoxymethyl)-2-methoxymethyl-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

According to the processes described in example 361 (a, b, c) and starting from 4-[4-((R)-2-ethoxy-propoxymethyl)-2-methoxymethyl-phenyl]-3,6-dihydro-2H-pyridine-1-carboxylic acid tert-butyl ester, the title compound is obtained as a yellow oil. Rf=0.78 (CH₂Cl₂/MeOH/conc. NH₃ 200:20:1); Rt=5.70 (gradient I).

### b) 4-[4-((R)-2-Ethoxy-propoxymethyl)-2-methoxymethyl-phenyl]-3,6-dihydro-2H-pyridine-1-carboxylic acid tert-butyl ester

To a solution of 8.37 mmol of 2-[4-((R)-2-ethoxy-propoxymethyl)-2-methoxy methyl-phenyl]-4,4,5,5-tetramethyl-[1,3,2]dioxaborolane, 9.21 mmol of 4-trifluoromethane-sulfonyloxy-3,6-dihydro-2H-pyridine-1-carboxylic acid tert-butyl ester [138647-49-1] and 25.11 mmol of LiCl in 50 mL of degassed DME under argon are added 20 mL of 2N NaHCO₃ solution, followed by 0.42 mmol of Pd(PPh₃)₄. The reaction is heated at 80° C. and stirred for 18 h. The mixture is cooled at RT, diluted with TBME, and washed with saturated aqueous NaHCO₃ solution. The aqueous layer is re-extracted 3× with TBME, the combined organic extracts are dried over Na₂SO₄, filtered and concentrated under reduced pressure. The residue is purified by flash chromatography (SiO₂ 60F) to afford the title compound as a light brown oil. Rf=0.25 (EtOAc/heptane 1:2); Rt=5.51 (gradient I).

### c) 2-[4-((R)-2-Ethoxy-propoxymethyl)-2-methoxy methyl-phenyl]-4,4,5,5-tetramethyl-[1,3,2]dioxaborolane

To a solution of 9.99 mmol of 1-bromo-4-((R)-2-ethoxy-propoxymethyl)-2-methoxy-methyl-benzene, 14.98 mmol of bis(pinacolato)diboron and 29.96 mmol of potassium acetate in 50 mL of degassed DMSO under argon are added 0.30 mmol of [1,1′-bis(diphenylphosphino)ferrocene]dichloro palladium(II). The reaction mixture is heated at 80° C., stirred for 18 h, then cooled to RT. The mixture is partitioned between H₂O and TBME and the aqueous phase is re-extracted with TBME (5×). The combined organic extracts are dried over Na₂SO₄, filtered and concentrated under reduced pressure. The residue is purified by flash chromatography (SiO₂ 60F), to afford the title compound as a pale green oil. Rf=0.36 (EtOAc/heptane 1:2); Rt=5.51 (gradient I).

### d) 1-Bromo-4-((R)-2-ethoxy-propoxymethyl)-2-methoxymethyl-benzene

According to the process described in example 373c, 1-bromo-4-chloromethyl-2-methoxymethyl-benzene is used to afford the title compound as a yellow oil. Rf=0.38 (EtOAc/heptane 1:2); Rt=5.07 (gradient I).

### e) 1-Bromo-4-chloromethyl-2-methoxymethyl-benzene

Analogously to the process described in example 374d, (4-bromo-3-methoxymethyl-phenyl)-methanol is used to afford the title compound as a yellow oil. Rf=0.73 (EtOAc/heptane 1:1); Rt=4.87 (gradient I).

### f) (4-Bromo-3-methoxymethyl-phenyl)-methanol

Analogously to the process described in example 374e, 4-bromo-3-methoxymethyl-benzoic acid methyl ester is used to afford the title compound as a colourless oil. Rf=0.15 (EtOAc/heptane 1:3); Rt=3.41 (gradient I).

### g) 4-Bromo-3-methoxymethyl-benzoic acid methyl ester

To a solution of 23.31 mmol of sodium methoxide in 20 mL of methanol at 0° C. is added dropwise a solution of 18.65 mmol of 4-bromo-3-bromomethyl-benzoic acid methyl ester [142031-67-2] in 10 mL of dry DMF. The reaction mixture is stirred at RT for 2 h, then at 50° C. for 20 h. The reaction mixture is cooled to RT, diluted with brine, and extracted with TBME (4×). The combined organic extracts are dried over Na₂SO₄ and concentrated under reduced pressure. The residue is purified by flash chromatography (SiO₂ 60F) to afford the title compound as a pale yellow oil. Rf=0.28 (EtOAc/heptane 1:3); Rt=4.45 (gradient I).

### Example 377

### (3S,4S)-4-[2-Ethoxymethyl-4-((R)-2-ethoxy-propoxymethyl)-phenyl]-3-[4-(3-methoxy-propyl)-3,4-dihydro-2H-benzo[1,4]oxazin-6-ylmethoxy]-piperidin-4-ol

According to general method A, (3S,4S)-4-[2-ethoxymethyl-4-((R)-2-ethoxy-propoxymethyl-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester is used to afford the title compound as a pale yellow oil. Rf=0.13 (CH₂Cl₂/MeOH/conc. NH₃ 200:10:1); Rt=4.11 (gradient I).

The starting materials are prepared as follows:

### a) (3S,4S)-4-[2-Ethoxymethyl-4-((R)-2-ethoxy-propoxymethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

According to the processes described in example 361 (a, b, c, d) and starting from 2-ethoxymethyl-4-((R)-2-ethoxy-propoxymethyl)-phenyl-boronic acid, the title compound is obtained as a colourless oil. Rf=0.29 (EtOAc/heptane 1:1); Rt=5.92 (gradient I).

### b) 2-Ethoxymethyl-4-((R)-2-ethoxy-propoxymethyl)-phenyl-boronic acid

According to the process described in example 361e, of 1-bromo-2-ethoxymethyl-4-((R)-2-ethoxy-propoxymethyl)-benzene is used to afford the title compound as a yellow oil. Rt=3.69 (gradient I).

### c) 1-Bromo-2-ethoxymethyl-4-((R)-2-ethoxy-propoxymethyl)-benzene

Analogously to the processes described for example 376 (d, e, f, g), and starting from of 4-bromo-3-bromomethyl-benzoic acid methyl ester [142031-67-2] and sodium ethoxide in step g, the title compound is obtained as a colourless oil. Rf=0.42 (EtOAc/heptane 1:2); Rt=5.44 (gradient I).

### Example 378

### (3S,4S)-4-[4-((R)-2-Ethoxy-propoxymethyl)-2-(2-methoxy-ethyl)-phenyl]-3-[4-(3-methoxy-propyl)-3,4-dihydro-2H-benzo[1,4]oxazin-6-ylmethoxy]-piperidin-4-ol

To a solution of 5.70 mmol of (3S,4S)-4-[4-((R)-2-ethoxy-propoxymethyl)-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol in 30 mL of methanol and 5 mL of tetrahydrofuran are added 28.48 mmol of sodium dihydrogen phosphate followed by 85.43 mmol of sodium amalgam (10% Na), in portions over 8 h. The reaction mixture is stirred for 18 h, diluted with H₂O and extracted with EtOAc (3×). The combined organic extracts are washed with brine, dried over Na₂SO₄ and concentrated under reduced pressure. The title compound is obtained from the residue by means of flash chromatography (SiO₂ 60F), to afford the title compound as a pale yellow oil. Rf=0.25 (CH₂Cl₂/MeOH/conc. NH₃ 200:20:1); Rt=3.99 (gradient I).

The starting materials are prepared as follows:

### a) (3S,4S)-4-[4-((R)-2-ethoxy-propoxymethyl)-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol

Analogously to the process described for example 373c, (3S,4S)-4-[4-chloromethyl-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol is used to afford the title compound as a pale yellow oil. Rf=0.23 (EtOAc/heptane 1:1); Rt=5.76 (gradient I).

### b) (3S,4S)-4-[4-Chloromethyl-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol

Analogously to the process described for example 374d, (3S,4S)-4-[4-hydroxymethyl-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol is used to afford the title compound as a yellow oil. Rf=0.53 (EtOAc/heptane 2:1); Rt=5.51 (gradient I).

### c) (3S,4S)-4-[4-Hydroxymethyl-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol

To a solution of 10.5 mmol of 4-[(3S,4S)-4-hydroxy-3-[4-(3-methoxy-propyl)-3,4-dihydro-2H-benzo[1,4]oxazin-6-ylmethoxy]-1-(toluene-4-sulfonyl)-piperidin-4-yl]-3-(2-methoxy-ethyl)-benzoic acid in 80 mL of THF are added 31.49 mmol of borane-THF complex (1M in THF). The reaction mixture is heated at 45° C. and stirred for 4 h. Once cooled to RT, 40 mL of MeOH are carefully added, and the mixture is concentrated under reduced pressure. The residue is purified by flash chromatography (SiO₂ 60F) to afford the title compound as a yellow oil. Rf=0.19 (EtOAc/heptane 4:1); Rt=4.73 (gradient I).

### d) 4-[(3S,4S)-4-Hydroxy-3-[4-(3-methoxy-propyl)-3,4-dihydro-2H-benzo[1,4]oxazin-6-ylmethoxy]-1-(toluene-4-sulfonyl)-piperidin-4-yl]-3-(2-methoxy-ethyl)-benzoic acid

A solution of 10.25 mmol of 4-[(3S,4S)-4-hydroxy-3-[4-(3-methoxy-propyl)-3,4-dihydro-2H-benzo[1,4]oxazin-6-ylmethoxy]-1-(toluene-4-sulfonyl)-piperidin-4-yl]-3-(2-methoxy-ethyl)-benzonitrile in 40 mL of EtOH, 10 mL of THF and 50 mL of 4N aqueous NaOH solution is stirred at 80° C. for 48 h. The reaction mixture is cooled to 0° C., and its pH is brought to pH 1 by addition of 4N aqueous HCl solution. The aqueous solution is extracted with EtOAc (3×), the combined organic extracts are washed sequentially with water and with brine, dried over Na₂SO₄ and concentrated under reduced pressure, to afford the crude title compound as a brown foam. Rf=0.05 (EtOAc/heptane 2:1); Rt=4.71 (gradient I).

### e) 4-[(3S,4S)-4-Hydroxy-3-[4-(3-methoxy-propyl)-3,4-dihydro-2H-benzo[1,4]oxazin-6-ylmethoxy]-1-(toluene-4-sulfonyl)-piperidin-4-yl]-3-(2-methoxy-ethyl)-benzonitrile

To a solution of 10.57 mmol of (3S,4S)-4-[4-chloro-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol in 30 mL of DMA at RT are added 13.75 mmol of Zn(CN)₂, 3.17 mmol of Pd₂(dba)₃ and 6.34 mmol of dppf. The reaction mixture is stirred at 140° C. for 48 h, cooled to ca. 60° C., and concentrated under reduced pressure. The residue is purified by flash chromatography (SiO₂ 60F) to afford the title compound as a light brown oil. Rf=0.29 (EtOAc/heptane 1:1); Rt=5.26 (gradient I).

### f) (3S,4S)-4-[4-Chloro-2-(2-methoxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol

To a solution of 12.43 mmol of (3S,4S)-4-[4-chloro-2-(2-methoxy-ethyl)-phenyl]-3-[4-(3-methoxy-propyl)-3,4-dihydro-2H-benzo[1,4]oxazin-6-ylmethoxy]-piperidin-4-ol in 120 mL of EtOAc and 120 mL of 2M aqueous Na₂CO₃ solution are added 13.06 mmol of 4-toluenesulfonyl chloride. The reaction mixture is stirred at RT for 4 h. The phases are separated and the aqueous phase is extracted with EtOAc (2×). The combined organic extracts are dried over Na₂SO₄ and concentrated under reduced pressure. The residue is purified by flash chromatography (SiO₂ 60F) to afford the title compound as a pale yellow foam. Rf=0.36 (EtOAc/heptane 1:1); Rt=5.70 (gradient I).

### g) (3S,4S)-4-[4-Chloro-2-(2-methoxy-ethyl)-phenyl]-3-[4-(3-methoxy-propyl)-3,4-dihydro-2H-benzo[1,4]oxazin-6-ylmethoxy]-piperidin-4-ol

According to general method A, (3S,4S)-4-[4-chloro-2-(2-methoxy-ethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.16 (CH₂Cl₂/MeOH/conc. NH₃ 200:20:1); Rt=3.93 (gradient I).

### h) (3S,4S)-4-[4-Chloro-2-(2-methoxy-ethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

According to the processes described in example 361 (a, b, c, d) and starting from 4-chloro-2-(2-methoxy-ethyl)-phenyl-boronic acid, the title compound is obtained as a colourless resin. Rf=0.18 (EtOAc/heptane 1:1); Rt=5.76 (gradient I).

### i) 4-Chloro-2-(2-methoxy-ethyl)-phenyl-boronic acid

According to the process described in example 361e, 1-bromo-4-chloro-2-(2-methoxy-ethyl)-benzene is used to afford the title compound as a yellow oil. Rf=0.31 (EtOAc/heptane 1:1); Rt=3.63 (gradient I).

### k) 1-Bromo-4-chloro-2-(2-methoxy-ethyl)-benzene

To a solution of 57.24 mmol of 2-(2-bromo-5-chloro-phenyl)-ethanol [947614-94-0] in 86 mL of DMF at 0° C. are added portionwise 60.1 mmol of NaH (55% dispersion in oil). The reaction mixture is stirred at 0° C. for 1 h, then 60.1 mmol of dimethyl sulfate are added, and the reaction mixture is stirred for 30 min, then diluted with TBME and quenched with 1N aqueous NaHCO₃ solution. The aqueous layer is re-extracted with TBME. The combined organic extracts are washed sequentially with water (2×) and brine, dried over Na₂SO₄ and concentrated under reduced pressure. The residue is purified by flash chromatography (SiO₂ 60F) to afford the title compound as a yellow oil. Rf=0.65 (EtOAc/heptane 1:3); Rt=5.19 (gradient I).

1. Compound of the general formula (I) 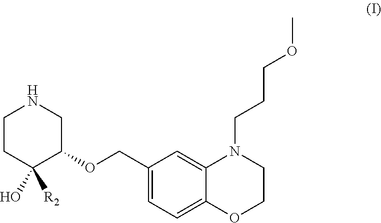
or a pharmaceutically acceptable salt thereof, in which R² is phenyl, which is substituted by 1-3 radicals, one of which is located in the para-position relative to the bond from the phenyl ring to the remainder of the molecule, selected independently from the group consisting of
C₁₋₆-alkanoyloxy-C₁₋₆-alkyl,
C₂₋₆-alkenyl,
C₂₋₆alkenyloxy,
C₂₋₆-alkenyloxy-C₁₋₆-alkyl,
C₁₋₆alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkoxy-C₁₋₆-alkyl,
C₁₋₆-alkoxy-C₁₋₆-alkylamino-C₁₋₆-alkyl,
C₁₋₆-alkoxy-C₁₋₆-alkylsulfanyl,
C₁₋₆-alkoxy-C₁₋₆-alkylsulfanyl-C₁₋₈-alkyl,
C₁₋₆-alkoxycarbonyl,
C₁₋₆-alkoxycarbonyloxy-C₁₋₈-alkyl,
C₁₋₆-alkyl,
C₁₋₈-alkylsulfanyl,
C₁₋₆-alkylsulfanyl-C₁₋₆-alkoxy,
C₁₋₆-alkylsulfanyl-C₁₋₆-alkoxy-C₁₋₆-alkyl,
C₁₋₆-alkylsulfanyl-C₁₋₆-alkyl,
C₁₋₆-alkylsulfonyl-C₁₋₆-alkoxy-C₁₋₆-alkyl,
C₁₋₆-alkylsulfonyl-C₁₋₆-alkyl,
C₂₋₈-alkynyl,
optionally N-mono- or N,N-di-C₁₋₆-alkylated amino-C₁₋₆-alkoxy,
optionally N-mono- or N,N-di-C₁₋₆-alkylated amino-carbonyl-C₁₋₆-alkyl,
aryl-pyrrolidinyl-C₀₋₈-alkoxy,
heterocyclyl-pyrrolidinyl-C₀₋₆-alkoxy,
aryl-C₀₋₆-alkoxy-C₁₋₆-alkoxy,
aryl-C₀₋₆-alkoxy-C₁₋₆-alkoxy-C₁₋₆-alkyl,
carboxy-C₁₋₆-alkyl,
cyano,
cyano-C₁₋₆-alkyl,
C₃₋₈-cycloalkyl-C₀₋₆-alkoxy-C₁₋₆-alkoxy,
C₃₋₈-cycloalkyl-C₀₋₆-alkoxy-C₁₋₆-alkoxy-C₁₋₆-alkyl,
C₃₋₈-cycloalkyl-C₀₋₆-alkoxy-C₁₋₆-alkyl,
C₃₋₈-cycloalkyl-C₀₋₆-alkylamino-C₁₋₆-alkyl,
heterocyclyl-carbonyl-C₁₋₆-alkyl,
heterocyclyl-sulfanyl-C₁₋₆-alkoxy-C₁₋₆-alkyl and
heterocyclyl-C₂₋₆-alkoxy-C₁₋₆-alkyl;
and may, in addition to the aforementioned substituents, also be substituted by a maximum of 2 halogens, the maximum total number of substituents on the phenyl radical of R² being 3.

2. Compound according to claim 1, in which R² is phenyl, substituted by 1-3 radicals, one of which is located in the para-position relative to the bond from the phenyl ring to the remainder of the molecule, selected independently from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkoxy-C₁₋₆-alkyl,
C₁₋₆-alkoxy-C₁₋₆-alkylsulfanyl,
C₁₋₆-alkoxy-C₁₋₆-alkylsulfanyl-C₁₋₆-alkyl,
C₁₋₆-alkyl,
C₁₋₆-alkylsulfanyl-C₁₋₆-alkoxy,
C₁₋₆-alkylsulfanyl-C₁₋₆-alkoxy-C₁₋₆-alkyl,
aryl-pyrrolidinyl-C₀₋₆-alkoxyl,
C₃₋₈-cycloalkyl-C₀₋₆-alkoxy-C₁₋₆-alkyl,
heterocyclyl-C₂₋₆-alkoxy-C₁₋₆-alkyl and
heterocyclyl-pyrrolidinyl-C₀₋₆-alkoxy, or a pharmaceutically acceptable salt thereof.

3. Compound according to claim 1, in which R² is phenyl, substituted by 1-2 radicals, one of which is located in the para-position relative to the bond from the phenyl ring to the remainder of the molecule, selected independently from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl.
C₁₋₆-alkoxy-C₁₋₆-alkyl,
C₁₋₆-alkyl,
C₃₋₈-cycloalkyl-C₀₋₆-alkoxy-C₁₋₆-alkyl,
heterocyclyl-C₂₋₆-alkoxy-C₁₋₆-alkyl and
heterocyclyl-pyrrolidinyl-C₀₋₆-alkoxy, or a pharmaceutically acceptable salt thereof.

4. Compound according to claim 1, in which R² is phenyl, para-substituted relative to the bond from the phenyl ring to the remainder of the molecule by 1 radical selected from the group consisting of
C₁₋₆-alkoxy,
C₁₋₆-alkoxy-C₁₋₆-alkoxy,
C₁₋₆-alkoxy-C₁₋₆-alkoxy-C₁₋₆-alkoxy,
C₁₋₆-alkoxy-C₁₋₆-alkoxy-C₁₋₆-alkoxy-C₁₋₆-alkyl,
C₁₋₆-alkoxy-C₁₋₆-alkoxy-C₁₋₆-alkyl,
C₁₋₆-alkoxy-C₁₋₆-alkyl,
C₁₋₆-alkyl,
C₃₋₈-cycloalkyl-C₀₋₆-alkoxy-C₁₋₆-alkyl,
heterocyclyl-C₂₋₆-alkoxy-C₁₋₆-alkyl and
heterocyclyl-pyrrolidinyl-C₀₋₈-alkoxy, or a pharmaceutically acceptable salt thereof.

5. Compound according to claim 1, in which a second substituent on the phenyl ring, when present, is located in the ortho position relative to the bond from the phenyl ring to the remainder of the molecule, or a pharmaceutically acceptable salt thereof.

6-7. (canceled)

8. Method for preventing, for delaying the progression of or for treating high blood pressure, heart failure, glaucoma, myocardial infarction, renal failure, restenoses or stroke in a patient, comprising administering to said patient a therapeutically-effective amount of a compound of formula (I) or a pharmaceutically acceptable salt thereof, according to claim 1.

9. Pharmaceutical product comprising a compound of the general formula (I) or a pharmaceutically acceptable salt thereof, according to claim 1, and conventional excipients.

10. A pharmaceutical combination in the form of a product or of a kit composed of individual components consisting a) of a compound of the general formula (I) or a pharmaceutically acceptable salt thereof, according to claim 1, and b) at least one pharmaceutical form as active ingredient having a cardiovascular effect.
